(19) United States
(12) Patent Application Publication
(10) Pub. No.: US 2004/0091448 A1
(43) Pub. Date: May 13, 2004

(54) Disinfecting dip compositions and related methods

(76) Inventor: Robert D. Kross, Bellmore, NY (US)

Correspondence Address: H.D. Coleman, Coleman Sudol Sapone, P.C., 714 Colorado Avenue, Bridgeport, CT, 06605-1601, US

(21) Appl. No.: 10/293,805

(22) Filed: Nov. 13, 2002

(51) Int. Cl. A61K 31/14

(52) U.S. Cl. 424/70.24; 514/642; 514/643

(57) ABSTRACT

The invention provides aqueous disinfecting (germicidal) dip compositions comprising opacifying complexes formed by the reaction of cationic organic quaternary ammonium compounds (“quats”)and anionic organic sulfonates. The compositions are particularly useful as dairy cow teat dips. Complexes formed by the reaction of quats and organic sulfonates provide turbidity and opacity to these aqueous germicidal dip compositions. Such turbidity and opacity serve to enhance the visibility of the compositions, e.g., when applied to an animal's teat.

### FIELD OF THE INVENTION

The invention provides aqueous disinfecting (germicidal) dip compositions comprising opacifying complexes formed by the reaction of cationic organic quaternary ammonium compounds (“quats”)and anionic organic sulfonates. The compositions are particularly useful as dairy cow teat dips. Complexes formed by the reaction of quats and organic sulfonates provide turbidity and opacity to these aqueous germicidal dip compositions. Such turbidity and opacity serve to enhance the visibility of the compositions, e.g., when applied to an animal's teat. Germicidal quats can be used to make the opacifying complexes employed in the compositions of the instant invention, and non-quat germicides and antimicrobial agents can also be used in the compositions. Other materials such as thickening agents, humectants, colorants and wetting agents may be included in the compositions.

The invention also provides methods for making an aqueous germicidal teat dip composition comprising opacifying complexes by combining individual components containing quats and organic sulfonates that react upon combination to form opacifying complexes. The invention further provides methods for disinfecting a substrate by contacting the substrate with a germicidal dip composition of the instant invention.

The invention also includes embodiments in which two or more compositions containing nongermicidal quats and organic sulfonates, when combined to make a dip, form (1) an opacifying complex within the dip from the reaction of the quats and organic sulfonates, and (2) a germicidal agent from the reaction of other ingredients in the compositions.

### BACKGROUND OF THE INVENTION

Skin is particularly susceptible to damage under adverse ambient conditions, such as windy, wet, and cold weather, when chapping most often occurs. Irritated, chapped and cracked skin tissue can readily harbor pathogenic bacteria, including those that cause mammalian mastitis. Sore mammalian teats are particularly sensitive to the attachment of the milking claws, which often limits the ability of dairymen to milk the affected quarter.

Mastitis is by far the most prevalent and costly disease affecting dairy herds and principally affects cows. At least half of the dairy animal population is thought to be affected by bovine mastitis to some degree. Mastitis causes a lowering of milk output and a reduced milk quality, accounting for losses in the U.S. alone approaching $2 billion, a major portion of which results from the lowered milk output of infected cows. Mastitis is an inflammation of the mammary gland, which can result from injury, but most often is principally caused by invasion of bacteria through the teat orifice during activities related to milking . Contagious microorganisms on contaminated equipment and hands can be transferred to the teat during the milking process, and thereafter can be transferred from cow to cow or cow to human to cow. Alternatively, environmental microorganisms deposit on the teat and udder between milking periods when the teat orifice remains open post-milking. There are primarily two contagious organisms that infect the teat, Staphylococcus aureus and Streptococcus agalactiae. Environmental organisms that infect the teat include Streptococcus uberis, Streptococcus dysgalactiae, Klebsiella pneumoniae and Escherichia coli; these are present in the cow's surroundings, including the soil, its bedding, feces, and contaminated water.

Treatment of mastitis is costly, often ineffective, and involves antibiotic therapy. The cow's milk must be discarded during treatment periods and for a number of days thereafter. Washing or disinfecting teats before milking, and dipping the teats afterwards, can markedly reduce the transfer by self-infection of environmental organisms to the teat. Washing or disinfecting teats post-milking with teat dips controls the transfer of contagious organisms from equipment and hands. Without such disinfection, the individual teat quarters may become mastitic, which causes problems ranging from lower milk quality and poorer milk yields to death of the afflicted animal.

Teat dips generally embody antimicrobial agents which reduce or destroy all such pathogens, and may contain humectants, thickening and/or barrier-forming agents, and colorants. The latter are included so that the teat dip deposit may be observed on the treated teat; such observation is necessary to determine which teats have been appropriately sanitized during the milking process.

Teat dips are usually liquid compositions, and are most often single solutions or suspensions used directly by dairymen by withdrawal of a day's dip portion from a large container. In recent years, two-part teat dips have also been employed, wherein the active antimicrobial agent is formed by combination of certain chemical compounds in both parts shortly before application. The antimicrobial agents most often found in single part systems include iodophors, quaternary ammonium compounds, organic sulfonates, and chlorhexidine. Two-part systems, most often, are based on the generation of chlorous acid and/or chlorine dioxide by combination of a metal chlorite in one part and an acid source in the other.

The visibility, i.e. detectability, of the teat dip deposit is aided by the presence of a colorant in the formulation, and such visibility is directly related to the concentration and the spectral characteristics of the colorant. Film thickness also plays a role in detection of the teat dip film; the thicker the light path, the greater the ease of detection. The thickness of the liquid film that remains after pre- or post-milking application of the teat dip can range from about 0.5 mm to 1-2 mm, depending upon the viscosity and related thixotropy of the dip. Thickening agents act to increase the amount of the teat dip composition deposited onto the teat surface. But, even with thick film teat deposits, and inclusion of naturally brown-colored iodophor teat dips, the presence of the colored film is often difficult to detect visually, particularly when the teat of the animal is dark or variegated in appearance.

Visualization of the deposited dip can be significantly enhanced by the inclusion of opacifiers, which lower the light transmission through the teat film and allow the scattered light from colored film to be better observed. U.S. Pat. No. 4,891,216 (“'216 Patent”) discloses the use of an insoluble silica opacifier and particulate titanium dioxide opacifier to make thickened, barrier-forming teat dips more visible. However, such mineral materials have a higher density than that of the aqueous dips, resulting in their gradual separation from the teat dip matrix between the time of production and use. As a result, the settling out of these opacifiers in germicide containers reduces their intended effectiveness in the product when applied to the teat. This is particularly true for non-thickened, low-viscosity teat dips, wherein the matrix is more inimical to the creation of stable mineral suspensions. A further problem associated with inorganic opacifiers is that the dairyman will often observe precipitates of these minerals at the bottom of the dip containers, leading him to question its nature and the stability of the product he is using on the animals' teats. Further, dairymen will often see the dried, generally—whitish mineral deposits on the animals' teats and mistake such deposits for an adverse skin condition resulting from the use of a particular teat dip.

The need exists, therefore, for disinfecting dip compositions, particularly teat dips, comprising opacifying agents which are stable from the time of production through the time of use and which enhance dip visibility. Further, the need exists for highly visible teat dips that may or not include color agents. Ideally, such compositions could be deposited as a thin or thick film on a treated teat and would contribute to the antimicrobial efficacy of the dip.

### OBJECTS OF THE INVENTION

It is an object of the present invention to provide aqueous disinfecting (germicidal) dip compositions comprising opacifying complexes which enhance the visibility of the dips following application to a substrate (as defined hereinafter).

It is a further object of the present invention to provide aqueous disinfecting (germicidal) teat dip compositions comprising opacifying complexes which enhance the visibility of the dips following application and which intensify the perceived color of a teat dip film deposited onto an animal's skin.

It is a further object of the present invention to provide disinfecting (germicidal) teat dip compositions comprising opacifying complexes that are stable and that are relatively free of the settling problems attendant to known particulate, inorganic opacifiers.

It is a further object of the present invention to provide disinfecting (germicidal) teat dip compositions comprising opacifying complexes in which the opacifying complexes are formed upon combination of the individual components or systems from which the dips are made.

It is a still further object of the present invention to provide an aqueous germicidal teat dip composition comprising an opacifying complex in which a germicidal component of the opacifying complex either enhances, or provides the sole source of, the dip's germicidal properties.

It is a further object of the present invention to provide methods for making an aqueous germicidal teat dip composition comprising opacifying complexes by combining individual components containing quats and organic sulfonates that react to form opacifying complexes.

It is a still further object of the present invention to provide methods for disinfecting a substrate by contacting the substrate with a germicidal teat dip composition of the instant invention.

### SUMMARY OF THE INVENTION

In accordance with the above-stated objects, the instant invention provides aqueous germicidal dip compositions and methods of using such compositions. Compositions of the instant invention have a particular utility as teat dips and are readily visible on a substrate upon application through standard means such as dipping or spraying. Opacifying complexes used in the compositions are stable prior to, during, and after application, unlike known teat dip formulations in which suspended, opacifying mineral particulates gradually separate during storage.

The compositions of the instant invention comprise stable, opacifying complexes formed by ionic bonding of cationic, organic quaternary ammonium compounds (“quats”) and anionic organic sulfonates, which can be a FD&C dye particularly suitable for teat dip applications. These complexes generally remain suspended indefinitely in the teat dip liquid medium and impart a turbidity and opacity to that medium. The quat cationic ammonium groups interact with the sulfonate groups so as to form ionic bonds. While it is not fully understood how the cationic moiety of the quaternary ammonium and the anionic moieties of the anionic organic molecules interact, and without in any way intending to be bound by theory, it does appear that the quaternary ammonium compound has a greater affinity for the anionic dye than for the anionic counter-ion to which the quat would generally bind. The same holds true for the anionic dye, which has more of an affinity for the cationic quat than for the cationic counter-ion.

The degree of turbidity of the compositions of the instant invention is directly related to the amount of ionic complex present, and the dip appearance can range from slightly hazy to overtly opaque depending upon concentration of the opacifying complex. Upon application of germicidal teat dip compositions of the instant invention through conventional means such as dipping or spraying, scattering of reflected, natural, or artificial light from the teat film occurs at multiple layers in the film, as opposed to the single reflection of incident light from the teat skin. Compositions that serve as opacifying complexes in the dips of the instant invention are described for unrelated uses in, for example, U.S. Pat. No. 5,948,152 (“'152 Patent”) and U.S. Pat. No. 5,938,828 (“'828 Patent”), the complete disclosures of which are hereby incorporated by reference. Preferred compositions of the instant invention also include antimicrobial agents and germicides. Other materials such as thickening agents, colorants, humectants and wetting agents may be included in the compositions of the instant invention.

The quaternary ammonium compound used in making opacifying complexes employed in the instant invention may be germicidal, e.g., a member of the class of monoalkyltrimethyl ammonium salts, monoalkyldimethyl benzyl ammonium salts, dialkyldimethyl ammonium salts, heteroaromatic ammonium salts, polysubstituted quaternary ammonium salts, bis-quaternary ammonium salts, polymeric quaternary ammonium salts, and imidazolinium quaternary ammonium salts. Thus, compositions of the instant invention include those in which germicidal action is provided solely by the quat used to make the aforementioned opacifying complex, and those in which another germicidal agent is included as a separate ingredient.

Quats useful in making the opacifying complexes used in compositions of the instant invention include those described in the '152 Patent and the '828 Patent and include the products of the nucleophilic substitution reaction of alkyl halides with tertiary amines. (Quats have four carbon atoms linked directly to the nitrogen atom; the anion of the alkylating agent is electrovalently bound to nitrogen.). Organic sulfonates that react which such quats to make the opacifying complexes include those soluble, anionic dyes having a sulfonic functionality disclosed in the '152 Patent and '828 Patent, as well as soluble dyes including members of the class of azo- or triphenylmethane-dyes which have established utility as teat dip colorizers. Examples of such dyes include FD&C Red #2, Yellow #5, Yellow #6, Red #4, Blue #1, Green #3 or Violet #1. These dyes color the compositions of the instant invention and make them more visible upon application. Organic sulfonates also include certain sulfonate polymers which have been used as thickeners in the teat dip field.

The instant invention also provides compositions and related methods in which quat and organic sulfonates are incorporated in either one- or two-part teat dip germicidal formulations to create turbid solutions which visually accentuate the presence of the teat dip film formed on the animals' teats following dipping or spraying. In one embodiment, an otherwise transparent germicidal teat dip, comprising one or more quat(s) as the sole germicidal agent(s), is combined with a sulfonate-containing colorant solution, thereby creating a stable, turbid solution having the same color as that of the colorant solution. In another embodiment, a transparent first part comprising an organic sulfonate dye and a transparent second part comprising a germicidal quat become sufficiently turbid upon combination so as to enhance the visual detectability of the applied dip. In another embodiment, a transparent first part comprising a germicidal quat colored with a non-sulfonate dye, and a transparent second part comprising a sulfonate present as a component of a thickening AMPS-based polymer, are combined to form a turbid, colored teat dip with a viscosity of greater than about 25 cps. The invention also includes embodiments in which two or more compositions containing nongermicidal quats and organic sulfonates, when combined to make a dip, form an opacifying complex within the dip from the reaction of the quats and organic sulfonates and a germicidal agent from the reaction of other ingredients in the compositions.

The amounts of quat and sulfonate materials used in the compositions and methods of the instant invention depend on the nature of the two materials, including such factors as molecular weights, solubilities, color intensity of the organic sulfonate, degree of turbidity produced by the selected ingredients, germicidal activity of the quat, and the thickening capacity of the sulfonate material. Such determinations can be readily made by those skilled in the art.

The invention also provides methods for making an aqueous germicidal teat dip composition comprising opacifying complexes by combining individual components containing quats and organic sulfonates that react to form opacifying complexes.

The invention further provides methods for disinfecting a substrate by contacting the substrate with a germicidal teat dip composition of the instant invention. In this context, the substrate may be any surface in need of such disinfection, including (but not limited to) skin or tissue in general, as well as body fluids and mucosal membranes. In a specific application, the substrate is the teat of a dairy cow.

Additional aspects of the instant invention are presented in the following detailed description.

### DETAILED DESCRIPTION OF THE INVENTION

As used herein, the following terms have the following respective meanings.

“Antimicrobial agents” include iodophors, quaternary ammonium compounds, chlorhexidine salts, chlorine release compounds (e.g. alkali hypochlorites), oxidizing compounds (e.g. hydrogen peroxide, peracids), protonated carboxylic acids (e.g. heptanoic, octanoic, nonanoic, decanoic, undecanoic acids), acid anionics (e.g. alkylaryl sulfonic acids), and chlorous acid and chlorine dioxide (from chlorite).

“Subject” or “patient” shall refer to a warm-blooded animal to which compositions according to the present invention may be applied. In certain preferred aspects according to the present invention, the preferred subject or patient is a cow, preferably a dairy cow.

“Germicides” which may be used in compositons of the instant invention include:

| | | |
|---|---|---|
| chlorhexidine gluconate | iodines and iodophors | octyl sulfonate |
| lauricidin | hydrogen peroxide | chlorine dioxide |
| quats | sodium hypochlorite | chlorous acid |
| lactic acid | linear dodecyl | capric acid |
| | benzene sulfonate | |
| bacteriocins | | caprylic acid |

“Cationic, organic quaternary ammonium compounds (‘quats’)” include those quats disclosed in the '152 Patent and '828 Patent. Preferred quats include monoalkyltrimethyl ammonium salts, monoalkyldimethyl benzyl ammonium salts, dialkyldimethyl ammonium salts, heteroaromatic ammonium salts, polysubstituted quaternary ammonium salts, bis-quaternary ammonium salts, polymeric quaternary ammonium salts, and imidazolinium quaternary ammonium salts. In a preferred embodiment, the opacifying complex used in the compositions of the instant invention is made with a quat that possesses antimicrobial activity, such that the quat supplements another germicide component or functions as the only germicide therein. Quat germicides can be categorized as follows: 1)-Monoalkyltrimethyl ammonium salts, 2)-monoalkyldimethyl benzyl ammonium salts, 3)-dialkyldimethyl ammonium salts, 4)-heteroaromatic ammonium salts, 5)-polysubstituted quaternary ammonium salts, 6) bis-quaternary ammonium salts, 7)-polymeric quaternary ammonium salts, and 8)-imidazolinium quaternary ammonium salts. Preferred examples include: 1)-CTAB [cetyltrimethyl ammonium bromide], 2)-Hyamine 3500, 3)-BTC 812 [octyldodecyldimethyl ammonium chloride], 4)-CPC [cetyl pyridinium chloride], 5)-Onyxide 172 [alkyldimethylethylbenzyl ammonium cyclohexylsulfamate, 6)-Dequadin [1,10-bis{2-methyl-4-aminoquinolinium chloride}-decane, 7)-Mirapol A-15 [Poly{N-[3-(dimethylammonio]propyl}-N′-[3-(ethyleneoxyethylene-dimethylammonio)propyl] urea dichloride] and imidazolinium quats [Methyl tallow amido-2-tallow imidazolinium methyl sulfate]. These are merely preferred germicidal quat compounds, and the invention contemplates the use of other germicidal compounds falling within the broadly listed classes of quats.

“Anionic organic sulfonates” or “organic sulfonates” include those anionic dyes having a sulfonic functionality disclosed in the '152 Patent and '828 Patent, as well as soluble dyes including members of the class of azo- or triphenylmethane-dyes. Examples of such dyes are those characterized as the Azo Dyes, including FD&C Red #2, Yellow #5, Yellow #6, Red #4, and those classified as the Triphenylmethane Dyes, including Blue #1, Green #3 or Violet #1. “Organic sulfonates” also include polymers with pendant sulfonate groups. A preferred group of such polymers are those derived from the AMPS (2-acrylamido-2-methylpropane sulfonate) monomer. The homopolymer of this material as well as certain AMPS copolymers with acrylamide and alkyl-substituted acrylamides have particular utility in both the formation of opacifying complexes and thickening of the dip formulation, and result in “barrier” film formation when dried on the teat. Other representative organic sulfonate compounds that are of value in this invention are: naphthalene 2-sulfonate polymer, as a latent opacifier and thickener; dodecyl sulfonate, as a latent opacifier; and Basyntan WL (BASF), a 25% aqueous solution of a poly(aromatic sulfonate)(i.e., phenolsulfonic acid) polymer, as a latent opacifier and thickener.

“Substrates” include the skin or tissue of a warm-blooded animal and, in a preferred embodiment, the teat of a dairy cow.

“Opacifying”, “opaque”, “turbid”, and “turbidity” are intended as relative terms that are not subject to precise measurement, but instead reflect a subjective assessment of the diminishment of light transparency through a composition. Thus, while “turbid” or “opacified” compositions are not completely clear (light transparent), they need not be completely opaque or impervious to light transmission. As mentioned, the dip appearance can range from slightly hazy to overtly opaque depending upon concentration of the opacifying complex.

“Pharmaceutically effective amount” as used herein denotes a quantity of the dip which, when administered to an irritated, cracked, or infected substrate, is sufficient to result in a measurable improvement in the condition of the substrate, including elimination of pathogens affecting the substrate adversely. The efficacy of dip treatment may be determined in a variety of ways, including visual detection. The term “effective amount” as used herein subsumes a pharmaceutically effective amount and denotes an amount of the dip or component of the dip which is used to effect its intended result.

In preferred compositions of the instant invention in which one or more quats serve as the only germicide(s), to preserve the germicidal efficacy of the teat dip, the amounts of such quat(s) used should be greater than those otherwise required to form an opacifying complex with a suitable organic sulfonate. In teat dips involving the use of a germicide other than the quat, equivalent stoichiometric amounts of the quat and the organic sulfonate can usually be used. When the organic sulfonate is a dye intended to impart a color to the teat dip, the complex of quat and dye will generally have the same perceived color as the uncomplexed dye, but will exist as a suspended complex molecule. Without in any way intending to be limited by theory, it appears that the complexation of the sulfonate moiety with the quaternary ammonium nitrogen moiety (the active site on the quat) does not ordinarily interfere with the electron resonance structure of the dye that provides color to the molecule. When the organic sulfonate is a non-dye germicidal sulfonate, the germicidal sulfonate's activity may be diminished by introducing a complexing quat that effectively eliminates a certain amount of the germicidal sulfonate. If the sulfonate is a thickener only, such as polymerized or copolymerized AMPS monomers, the thickening capacity of the polymer is minimally reduced by the presence of the opacifying quat, which is generally used at a much lower level than that of the AMPS polymer thickeners.

In two-part teat dip compositions of the instant invention, in which the active germicidal agent is created by combination of the two inactive or minimally-active parts of the teat dip, there are several options available to create opacified solutions in accordance with the invention: (1) opacify both parts of the inactive system, a priori, by including quat/organic sulfonate complexes in each part; (2) opacify one of the two parts, with the remaining part being transparent, where the combination of the two then becomes opacified; (3) do not opacify either part, but instead include one of the components of the potential quat-organic sulfonate complex in one part and one in the other, such that the combination becomes turbid as a result of the two parts being combined. When the organic sulfonate is not a dye, and no other dyes are included in the composition, the opacity of the solution (in either a one- or two-part system) will assume a whitish coloration from the scattering of incident light. Inherently colored teat dips, such as iodine dips, are an exception; a brown turbid teat dip film would be created. The use of a sulfonate-based dye as a source of the sulfonate group would introduce that color to the teat dip as well as to the film resulting from its application to the animal.

The amount of quat used in making the opacifying complexes included in compositions of the instant invention is dependent on such factors as the molecular weight of the compound, the turbidity of the solution it forms when combined with a specific organic sulfonate, the desired degree of turbidity, and whether the quat has germicidal properties which are desired for inclusion in the particular dip. In the latter case, a determination must be made a priori of the amount of quat that is germicidally-inactivated by combination with the desired level of sulfonate, such that an excess of that quat is needed in order to provide the required germicidal action. The effect of the quat on dip surface tension should also be considered, since many quats are surfactants. Generally, effective levels of quat used in the germicidal solution fall within the in the range of about 0.002% to about 2.0% by weight or higher (preferably no more than about 1.0% within this range), more preferably about 0.008% to about 0.5% by weight, and even more preferably in the range of 0.01% to about 0.2% by weight of the solution.

The amount of the organic sulfonate materials used is dependent on factors such as the nature of the organic sulfonate, including whether or not the sulfonate group is a component of a molecule containing a colorant moiety. If the sulfonate compound is a colorant, the nature and intensity of the colored complex formed with the selected quat should be considered. If the sulfonate compound is not a colorant, the nature and opacification tendency of the complex formed with the selected quat should be evaluated. If the sulfonate is a component of a polymer, the degree of thickening imparted by the sulfonate compound, and the level of sulfonate in that polymer, should be evaluated. Another factor relating to the quantity of total organic sulfonate used in making the compositions is whether the organic sulfonate functions solely as a component of the opacifying complex or, instead, serves another function. For example a sulfonated AMPS polymer, or copolymer, e.g., with acrylamide monomer, will generally be included at levels significantly above those necessary for complexation with the sulfonate moiety. While the level of polymer solids in the active formulation may approximate, say, 4%-5% by weight, the amount of sulfonated polymer required for the desired opacification may be no greater than, say, 0.2% by weight.

If the organic sulfonate is a colorant that imparts a significant color to the formula when used in small amounts, the sulfonate levels range from about 0.005% to about 3% in the active formulation, in both a one-part teat dip or a mixed two-part teat dip. Preferable levels of colorant are in the range of about 0.01% to about 2% by weight. The amount used depends upon the inherent intensity of the specific colorant and the preferred color of the teat dip solution.

Alcohol-containing humectants or antifreezes may also be included in compositions of the instant invention. These include monohydroxy and polyhydroxy alcohols, and certain alkyl ethers of polyhydroxy alcohols. Concentrations of such componenets range from about 2% to 40% by weight, generally from 3% to 25% and typically from 5% to 15% by weight. Representative monohydroxy alcohols are ethyl alcohol, n-propanol and isopropanol, and representative polyhydroxy alcohols or alkyl ethers thereof are glycerin, sorbitol, 1,2- and 1,3-propylene glycol, dipropylene glycol, alkyl ethers of dipropylene glycols, other sugar alcohols, and mixtures thereof. Preferred humectants are glycerin and sorbitol, which may be employed at a level of from about 0.5% to about 20% by weight, and more preferably from about 2% to about 10% by weight of the disinfecting composition.

Various optional ingredients may be included in compositions of the instant invention. Such ingredients include (but are not limited to) wetting agents, textural modifiers, and film-forming polymers. The wetting agents facilitate contact of the composition with the skin, and can be selected from those materials recognized to provide this effect, in both identity and amount. Textural modifiers are those materials which primarily affect the body of the mixed composition in terms of retention, flow and lubricity. These include thickening agents such as alkyl celluloses, alkoxy celluloses, xanthan gum, guar gum, and polyacrylamide derivatives, of which the polymer of 2-acrylamido-2-methylpropane sulfonic acid is a preferred example. Other textural modifiers include lanolin derivatives, acyl lactylates, polyethylene glycol, glyceryl esters, and mixtures thereof. Film-forming polymers include the above-referenced polyacrylamides, as well as the class of poly(vinyl alcohols/vinyl acetates) and polyvinyl pyrollidone.

The compositions of the instant invention may also include antimicrobial organic acids, including such small molecule acids as formic, acetic and propionic acids, among others, as well as members of the group of alpha-hydroxy carboxylic acids having a pKa from 2.8 to 4.2 such as glycolic, lactic, malic, mandelic, citric and tartaric acids. Other, weaker acids that may be used include benzoic, caprylic, capric and the hydroxybenzoic acids. Salts of these organic acids that may be used include potassium, sodium and quaternary ammonium salts. Preferred salts of organic acids include sodium or potassium lactate, mandelate, citrate or malate. Compositions of the instant invention can contain a salt of an organic acid at a concentration ranging from about 0.25% to about 10%, generally from 0.5% to 5% and typically from 1% to 3% by weight. As disclosed in U.S. Pat. No. 6,123,966, these salts of organic acids may be reacted in two-part systems with a metal chlorite to form germicidal agents. The metal chlorite is present in the second part of a two-part system such that, when combined with the first part, its level in the disinfecting composition ranges from 0.005-1% by weight, generally from 0.05-0.5% and typically from 0.1-0.4% by weight. Suitable metal chlorites are water-soluble chlorites, including alkali or alkaline earth chlorites, such as sodium or potassium chlorite. A base is also present in the second part in an amount sufficient to adjust the pH of the second part to a value ranging from about 9 to about 11.5, and typically from 10.5 to 11. Suitable bases useful in this regard include alkali metal hydroxides such as sodium, potassium or lithium hydroxide.

Formula optimization based on the aforementioned factors is within the skill of those in the teat dip formulation art. The invention is described further in the following examples, which are illustrative and not limiting.

### EXAMPLE 1

This example demonstrates how, in a teat dip of the instant invention, the amount of quat and organic sulfonate necessary to provide a desired turbidity or opacity was determined. In this example, the quat used was the germicide benzylammonium chloride [BAC] and the organic sulfonate used was an AMPS polymer, i.e., poly (2-acrylamido-2-methylpropane sulfonate, sodium salt). Varying concentrations of each component were combined, and the resulting white suspensions were screened in order to select the most desirable degree of opacification for a colorless teat dip based on the same germicide, i.e. benzylammonium chloride. Unless otherwise noted, all parts and percentages in this and the following examples, as well as in the instant disclosures and claims, are weight percentages.

The various concentrations of the two components, in the final mixture, were as follows:

Observations of mixtures of the BAC
quat and the AMPS polymer sulfonate

| | | poly | |
|---|---|---|---|
| Run | BAC (%) | (AMPS) (%) | Observation |
| A | 0.025 | 0.025 | Light haze |
| B | 0.083 | 0.017 | Similar to A |
| C | 0.067 | 0.033 | More turbid |
| D | 0.050 | 0.050 | Similar to C |
| E | 0.23 | 0.023 | Similar to A |
| F | 0.21 | 0.042 | Similar to C and D |
| G | 0.19 | 0.058 | Similar to C and D |
| H | 0.17 | 0.083 | Opaque |
| I | 0.13 | 0.130 | Similar to H |
| J | 0.91 | 0.091 | Similar to H and I |

These results indicate that the turbidity or opacity of the solution with the quat/sulfonate complex is largely determined by the amount of the polymer, and that quat levels can be significantly increased above a certain threshold without a further increase in opacity. It was thus concluded that approximately 0.05% of the BAC is inactivated in the quat/sulfonate complex, and that BAC above that level in solution is free to exert its germicidal effect. On that basis, a germicidal teat dip was formulated to contain 0.05% of the AMPS polymer and 1.05% of the benzalkonium chloride, so as to provide about 1.0% of uncomplexed, cidal BAC. The turbid teat film was more readily visible on the teat models to which it was applied than a similar formulation without the AMPS polymer. At that level of AMPS polymer use, there was no discernible increased viscosity of the solution.

### EXAMPLE 2

This example also demonstrates determination of the appropriate levels of both quat and organic sulfonate to provide a desired turbidity or opacity for a teat dip of the instant invention. In the experiments reflected in this example, the quat was the germicide benzylammonium chloride [BAC] and the organic sulfonate was the germicide dodecylbenzene sulfonic acid, sodium salt (DDBSA). Both materials have found use individually in previously marketed teat dips for teat dip disinfection. As opposed to Example 1, however, where that BAC in excess of the amount required to form the opacifying complex could serve as the source of germicidal action, the excess amount of the DDBSA was complexed with BAC, and the non-complexed material served as the source of antimicrobial efficacy. The various concentrations of the two components, in the final mixture, were as follows:

Observations of mixtures of the BAC
quat and the organic sulfonate DDBSA

| Run | DDBSA (%) | BAC (%) | Observation |
|---|---|---|---|
| A | 0.91 | 0.091 | Light haze |
| B | 0.83 | 0.17 | Moderate haze |
| C | 0.71 | 0.29 | Virtually opaque |
| D | 0.625 | 0.375 | Chalky white, |
| | | | with precipitate |

Accordingly, the teat dip was formulated with 1.0% DDBSA so as to contain about 0.7% of the active germicide DDBSA, and about 0.3% of the quat BAC to form the complex. In this formulation, the totally-complexed BAC provided no germicidal activity. The dip was quite evident on simulated teats that were used to evaluate the visibility of the resulting teat dip film.

### EXAMPLE 3

This example illustrates the preparation of a two-part chlorous acid/chlorine dioxide germicidal teat dip, each part of which is transparent, but which combine to create an opaque, colored germicidally-active composition. One part contained lactic acid and the quat benzylammonium chloride [BAC], and the other part contained sodium chlorite and dodecylbenzene sulfonic acid, sodium salt [DDBSA]. The formulas for each part were as follows:

| Part A | | |
|---|---|---|
| Lactic Acid- | 2.00% | (2.27% of 88% |
| | | tech. grade material) |
| BAC- | 0.30% | (1.76% of 17% |
| | | tech. grade material) |
| Pylaklor | 0.50% | |
| Permanent | | |
| Green S-722- | | |
| Water- | q.s. | |
| Part B: | | |
| Sodium Chlorite- | 1.00% | (1.22% of 82% |
| | | tech. grade material) |
| DDBSA- | 2.00% | (Sigma D-2525) |

| | |
|---|---|
| Water- | q.s. |

The perceptibility of the dip when deposited on a model teat was markedly enhanced by the opacification of the green teat-dip film.

### EXAMPLE 4

This example illustrates the preparation of a one part teat dip with a variety of organic acid and glyceryl monolaurate germicides, opacified with a complex of the quat dodecylbenzyldimethyl ammonium chloride [DDBDAC] and the organic sulfonate FD&C Yellow #5, which also imparts a color to the opaque solution. The opaque yellow suspension is stable, contains glycerin as a humectant, and is readily visible on animals' teats following application.

The composition of the opaque yellow dip is as follows:

| | |
|---|---|
| Glyceryl monolaurate- | 0.25% |
| Octanoic acid- | 0.625% |
| Decanoic acid- | 0.625% |
| Lactic acid (88% tech.)- | 1.70% |
| Glycerin- | 5.00% |
| FD&C Yellow #5- | 0.30% |
| Dodecylbenzyldimethyl | 0.15% |
| ammonium chloride- | |
| Water- | q.s. |

The dip is prepared by predissolving the octanoic and decanoic acids in a mixture of the glycerin and glyceryl monolaurate, adding the bulk of the water, followed by addition of the lactic acid and the yellow dye. The solution is stirred and brought to about 99% of its final weight with water, around 20% concentrate of the DDBDAC is then added to the solution sufficient to result in the final desired concentration, and the solution is then brought to the final weight with water.

It is to be understood by those skilled in the art that the foregoing description and examples are merely illustrative of the present invention, and should in no way be intrepreted as limiting the scope of the present invention. Variations of the detail presented herein may be made without departing from the spirit and scope of the present invention as defined by the following claims.

What is claimed is:

1. An aqueous germicidal dip composition comprising opacifying complexes formed by the reaction of an organic quaternary ammonium compound and an organic sulfonate.

2. The composition of claim 1, wherein the composition is a teat dip.

3. The composition of claim 1, wherein:
(a) the organic quaternary ammonium compound is selected from the group consisting of monoalkyltrimethyl ammonium salts, monoalkyldimethyl benzyl ammonium salts, dialkyldimethyl ammonium salts, heteroaromatic ammonium salts, polysubstituted quaternary ammonium salts, bis-quaternary ammonium salts, polymeric quaternary ammonium salts, polymeric quaternary ammonium salts, and imidazolinium quaternary ammonium salts; and
(b) the organic sulfonate is selected from the group consisting of azo- or triphenylmethane-dyes, a homopolymer or polymer derived from a monomer of AMPS (2-acrylamido-2-methylpropane sulfonate), copolymers of AMPS and acrylamide, copolymers of AMPS and alkyl-substituted acrylamides, naphthalene 2-sulfonate polymer, dodecyl sulfonate, Basyntan WL, and dodecylbenzene sulfonic acid sodium salt (DDBSA).

4. The composition of claim 1, wherein the organic quaternary ammonium compound is selected from the group consisting of CTAB (cetyltrimethyl ammonium bromide), Hyamine 3500, BTC 812 (octyldodecyldimethyl ammonium chloride), CPC (cetyl pyridinium chloride), Onyxide 172 (alkyldimethylethylbenzyl ammonium cyclohexylsulfamate), Dequadin (1,10-bis(2-methyl-4-aminoquinolinium chloride)-decane), Mirapol A-15 (Poly(N-(3-(dimethylammonio]propyl)-N′-)3-(ethyleneoxyethylene-dimethylammonio)propyl) urea dichloride), and imidazolinium quats (Methyl tallow amido-2-tallow imidazolinium methyl sulfate).

5. A composition of claims 2, 3, or 4, wherein said composition comprises one or more of the following: an antimicrobial agent that is not an organic quaternary ammonium compound, a germicide that is not an organic quaternary ammonium compound, a colorant or dye that is not an organic sulfonate, a pharmaceutical agent that is not an antimicrobial agent or a germicide, a humectant, an antifreeze, a wetting agent, a textural modifier, a film-forming polymer, an antimicrobial organic acid, and a metal chlorite.

6. The composition of claims 1 or 2, wherein the germicidal activity of the composition is attributable to the organic quaternary ammonium compound and an unreacted amount of organic quaternary ammonium compound is present in the composition.

7. The compositions of claims 1 or 2, wherein the germicidal activity of the composition is attributable to a component other than the organic quaternary ammonium compound.

8. The composition of claim 2, wherein the organic quaternary ammonium compound is benzyl ammonium chloride [BAC] and the organic sulfonate is the sodium salt of poly (2-acrylamido-2-methylpropane sulfonate).

9. The composition of claim 2, wherein the organic quaternary ammonium compound is benzylammonium chloride [BAC] and the organic sulfonate is dodecylbenzene sulfonic acid, sodium salt (DDBSA).

10. The composition of claim 2, wherein the organic quaternary ammonium compound is dodecylbenzyldimethyl ammonium chloride [DDBDAC] and the organic sulfonate is the dye FD&C Yellow #5.

11. The composition of claim 1, wherein the organic sulfonate is selected from the group consisting of the dyes FD&C Red #2, FD&C Yellow #5, FD&C Yellow #6, FD&C Red #4, FD&C Blue #1, FD&C Green #3, and FD&C Violet #1.

12. The compositions of claims 1, 2, or 3, comprising about 0.002% to about 1.0% by weight of unreacted organic quaternary ammonium compound and about 0.002% to about 1.0% by weight of unreacted organic sulfonate.

13. The composition of claim 2, wherein the opacifying complex is formed by the reaction of benzalkonium chloride and AMPS (2-acrylamido-2-methylpropane sulfonate) polymer, in an approximate ratio of around 20 to 25 parts benzalkonium chloride to around 1 part AMPS polymer by weight, and wherein around 0.5% to 1.5% by weight benzalkonium chloride remains unreacted in the composition.

14. The composition of claim 2, wherein the composition is a turbid, colored teat dip with a viscosity of greater than about 25 cps.

15. An aqueous, opacified germicidal dip made by combining a first composition comprising an organic quaternary ammonium compound and a second composition comprising an organic sulfonate
wherein said organic quaternary ammonium compound and organic sulfonate react upon combination to form a stable, opacifying complex.

16. The aqueous, opacified germicidal dip of claim 15, wherein said dip comprises an unreacted, germicidal amount of the organic quaternary ammonium compound.

17. The aqueous, opacified germicidal dip of claims 15 or 16, wherein the organic sulfonate is a soluble dye.

18. The aqueous, opacified germicidal dip of claims 15, wherein said dip comprises an unreacted, thickening amount of the organic sulfonate.

19. The aqueous, opacified germicidal dip of claims 15, wherein the organic sulfonate is a dip colorant.

20. The aqueous, opacified germicidal dip of claims 15, wherein the organic quaternary ammonium compound is a germicide.

21. The aqueous, opacified germicidal dip of claims 15, wherein the organic quaternary ammonium compound is the only germicidal component and the dip has the same color as the organic sulfonate.

22. The dip of claim 15, wherein the dip has the same color as a dye present in the first composition.

23. The dip of claim 22, wherein the organic sulfonate is a component of a thickening polymer.

24. The dip of claim 23, wherein the thickening polymer is an AMPS-based polymer.

25. An aqueous, opacified germicidal dip made by combining a first composition comprising an organic quaternary ammonium compound and a second composition comprising an organic sulfonate wherein (1) said organic quaternary ammonium compound and organic sulfonate react upon combination to form a stable, opacifying complex; and (2) ingredients in the first and second compositions other than the organic quaternary ammonium compound and organic sulfonate react upon combination to form a germicide.

26. The dip of claim 25, wherein the dip has the color of a dye present in the first composition.

27. The dip of claim 25, wherein the organic sulfonate is a dye that colors the dip.

28. The dip of claim 25, wherein the organic quaternary ammonium compound is a germicide.

29. The dip of claim 28, wherein an amount of the organic quaternary ammonium compound remains unreacted after combination of the first and second compositions.

30. An aqueous, opacified germicidal dip made by combining (1) an opacified first composition comprising an opacifying complex formed by the reaction of an organic quaternary ammonium compound and an organic sulfonate, and (2) a second, essentially translucent composition wherein ingredients in the first and second compositions other than the organic quaternary ammonium compound and organic sulfonate react upon combination to form a germicide.

31. The dip of claim 30, wherein the dip has the color of a dye present in the first composition.

32. The dip of claim 30, wherein the organic sulfonate is a dye that colors the dip.

33. The dip of claim 30, wherein the organic quaternary ammonium compound is a germicide.

34. The dip of claim 33, wherein an amount of the organic quaternary ammonium compound remains unreacted after combination of the first and second compositions.

35. An aqueous, opacified germicidal dip made by combining (1) an opacified first composition comprising an opacifying complex formed by the reaction of an organic quaternary ammonium compound and an organic sulfonate, and (2) an opacified second composition comprising an opacifying complex formed by the reaction of an organic quaternary ammonium compound and an organic sulfonate wherein ingredients in the first and second compositions other than the organic quaternary ammonium compound and organic sulfonate react upon combination to form a germicide.

36. The dip of claim 35, wherein the dip has the color of a dye present in the first composition.

37. The dip of claim 35, wherein the organic sulfonate is a dye that colors the dip.

38. The dip of claim 35, wherein the organic quaternary ammonium compound is a germicide.

39. The dip of claim 38, wherein an amount of the organic quaternary ammonium compound remains unreacted after combination of the first and second compositions.

40. A dip of claim 25, wherein the only germicidal agent present in the dip is formed upon combination of the first and second compositions.

41. A turbid, colored teat dip with a viscosity of greater than about 25 cps. made by combining a transparent first composition comprising a germicidal quat colored with a non-sulfonate dye and a transparent second composition comprising a thickening AMPS-based polymer having an an organic sulfonate moiety.

42. A dip of claim 25, wherein the germicidal agent formed upon combination of the first and second compositions is the reaction product of an organic acid and a metal chlorite.

43. A method of opacifying an aqueous germicidal dip composition comprising forming an opacifying complex in the dip by reacting an organic quaternary ammonium compound and an organic sulfonate in situ.

44. The method of claim 43, wherein:
(a) the organic quaternary ammonium compound is selected from the group consisting of monoalkyltrimethyl ammonium salts, monoalkyldimethyl benzyl ammonium salts, dialkyldimethyl ammonium salts, heteroaromatic ammonium salts, polysubstituted quaternary ammonium salts, bis-quaternary ammonium salts, polymeric quaternary ammonium salts, polymeric quaternary ammonium salts, and imidazolinium quaternary ammonium salts; and
(b) the organic sulfonate is selected from the group consisting of azo- or triphenylmethane-dyes, a homopolymer or polymer derived from a monomer of AMPS (2-acrylamido-2-methylpropane sulfonate), copolymers of AMPS and acrylamide, copolymers of AMPS and alkyl-substituted acrylamides, naphthalene 2-sulfonate polymer, dodecyl sulfonate, Basyntan WL, and dodecylbenzene sulfonic acid sodium salt (DDBSA).

45. The method of claim 43, wherein the organic quaternary ammonium compound is selected from the group consisting of CTAB (cetyltrimethyl ammonium bromide), Hyamine 3500, BTC 812 (octyldodecyldimethyl ammonium chloride), CPC (cetyl pyridinium chloride), Onyxide 172 (alkyldimethylethylbenzyl ammonium cyclohexylsulfamate), Dequadin (1,10-bis(2-methyl-4-aminoquinolinium chloride)-decane), Mirapol A-15 (Poly(N-(3-(dimethylammonio]propyl)-N′-)3-(ethyleneoxyethylene-dimethylammonio)propyl) urea dichloride), and imidazolinium quats (Methyl tallow amido-2-tallow imidazolinium methyl sulfate).

46. A method of making an opacified, aqueous germicidal dip comprising mixing aqueous solutions comprising an organic quaternary ammonium compound, an organic sulfonate, and one or more of the following: an antimicrobial agent which is not an organic quaternary ammonium compound, a germicide which is not an organic quaternary ammonium compound, a colorant or dye which is not an organic sulfonate, a pharmaceutical agent that is not a germicide, a humectant, an antifreeze, a wetting agent, a textural modifier, a film-forming polymer, an antimicrobial organic acid, or a metal chlorite.

47. The method of claim 46, wherein the dip has the color of a dye present in the first composition.

48. The method of claim 46, wherein the organic sulfonate is a dye that colors the dip.

49. The method of claim 46, wherein the organic quaternary ammonium compound is a germicide.

50. The method of claim 49, wherein an amount of the organic quaternary ammonium compound remains unreacted after combination of the first and second compositions.

51. A method of making an aqueous, opacified germicidal dip comprising combining: (1) an opacified first composition comprising an opacifying complex formed by the reaction of an organic quaternary ammonium compound and an organic sulfonate, and (2) a second, essentially translucent composition
wherein ingredients in the first and second compositions other than the organic quaternary ammonium compound and organic sulfonate react upon combination to form a germicide.

52. The method of claim 51, wherein the dip has the color of a dye present in the first composition.

53. The method of claim 51, wherein the organic sulfonate is a dye that colors the dip.

54. The method of claim 51, wherein the organic quaternary ammonium compound is a germicide.

55. The method of claim 54, wherein an amount of the organic quaternary ammonium compound remains unreacted after combination of the first and second compositions.

56. A method of making an aqueous, opacified germicidal dip comprising combining: (1) an opacified first composition comprising an opacifying complex formed by the reaction of an organic quaternary ammonium compound and an organic sulfonate, and (2) an opacified second composition comprising an opacifying complex formed by the reaction of an organic quaternary ammonium compound and an organic sulfonate
wherein ingredients in the first and second compositions other than the organic quaternary ammonium compound and organic sulfonate react upon combination to form a germicide.

57. The method of claim 56, wherein the dip has the color of a dye present in the first composition.

58. The method of claim 56, wherein the organic sulfonate is a dye that colors the dip.

59. The method of claim 56, wherein the organic quaternary ammonium compound is a germicide.

60. The method of claim 59, wherein an amount of the organic quaternary ammonium compound remains unreacted after combination of the first and second compositions.

61. The method of claim 46, wherein the only germicidal agent present in the dip is formed upon combination of the first and second compositions.

62. A method of treating a sore, irritated, chapped, cracked, or infected substrate, comprising applying a dip of claim 1, 3, or 4 to the substrate.

63. The method of claim 62, wherein the substrate is a mammalian teat.

64. A method of treating a mammal suffering from mastitis, comprising applying to the mammary gland of a mammal in need a pharmaceutically effective amount of a dip of claims 1, 3, or 4.

65. The method of claim 64, wherein the mammal is a cow.
